(19) United States
(12) Patent Application Publication
(10) Pub. No.: US 2009/0204076 A1
(43) Pub. Date: Aug. 13, 2009

(54) Medical Injector

(76) Inventor: Barry Peter LIVERSIDGE, Colchester (GB)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 12/426,679

(22) Filed: Apr. 20, 2009

Related U.S. Application Data
(63) Continuation-in-part of application No. 10/518,950, filed on Jun. 29, 2005.

(30) Foreign Application Priority Data
Feb. 3, 2003 (GB) 0302393.4
Jun. 23, 2003 (GB) PCT/GB2003/002689

(51) Int. Cl. A61M 5/32

(52) U.S. Cl. 604/196

(57) ABSTRACT

A medical injector has a syringe body 80 with a needle 81 secured at the forward end thereof and a plunger 82 projecting from the rearward end. The plunger 82 is arranged to drive a piston 83 forwardly into the syringe body 80 to dispense medicament through the needle. A safety device for the syringe includes a sleeve 89 slidably mounted on the syringe and spring loaded to a forward position where the sleeve 89 covers the projecting needle 81. An outwardly directed protuberance 84 is disposed partway along the length of the plunger 82 and engageable with the syringe body 80. The protuberance 84 is resiliently movable inwardly of the plunger 82 and defines a stop position for the plunger, subsequent increased pressure on the plunger 82 then allowing the plunger to move forwardly to dispense medicament. The protuberance 84 allows proper sequencing of the injector such that the needle 81 may penetrate a patient before medicament is dispensed.

### CROSS REFERENCE TO RELATED APPLICATIONS

This application is a continuation in part of U.S. patent application Ser. No. 10/518,950 filed on Jun. 29, 2005 which is based on International Patent Application No. PCT/GB2003/002689 filed on Jun. 23, 2003, which in turn claims the priority of UK Patent Application Nos. 0214452.5 and 0302393.4, filed respectively on Jun. 22, 2002 and Feb. 3, 2003 though this continuation in part application claims the priority solely of UK Patent Application No. 0302393.4 filed on Feb. 3, 2003. All of these said applications stand in the name of Barry Peter Liversidge.

### BACKGROUND OF THE INVENTION

a) Field of the Invention

This invention relates to a medical injector having a needle intended for penetration of a human or animal body, or for other medical uses such as the penetration of a pierceable membrane of an intravenous medication system. For convenience, in the following all such medical uses will be described simply as the penetration of a body, even though specific embodiments may be intended for other medical uses.

b) Description of the Prior Art

Fluids of various kinds may be administered to a human or animal body by means of a hollow needle in conjunction with a source of the required fluid. For example, such a needle may be mounted on a syringe pre-filled with a liquid drug, the needle being used to penetrate the body at the site at which the drug is to be received, the drug then being expelled from the syringe by applying force to the rear end of the syringe plunger.

It will be appreciated that a successful injection requires a correct sequence of events: firstly the insertion of the needle into a body to the correct depth followed by the complete delivery of the drug through the hollow needle and into the body. This required sequence has been attained by a variety of prior art devices, such as those described in the following summaries.

US2001/0005781-A1 describes a medical injector for use with a pre-filled syringe having a needle secured to the forward end thereof and in which there is a mechanism arranged selectively to drive forwardly the syringe and needle so as to penetrate a body and then to apply a force to the plunger so as to expel the drug from the syringe, thus completing the correct injection sequence. The medical injector has a safety device on the forward end thereof and including a sleeve slidable with respect to the needle, to protect the needle both before and after an injection. In operation, the medical injector mechanism is arranged push the syringe and needle forwardly while preventing forward movement of the plunger and piston until the needle has sufficiently penetrated a patient, whereafter the drug is dispensed through the needle. This proper sequencing of the injection is controlled by a structure within the injector that only allows movement of the syringe plunger and piston when the whole syringe has moved forwardly by a sufficient extent.

U.S. Pat. No. 3,797,489 discloses a similar arrangement for use with a pre-filled syringe wherein spring power drives an ampoule and cannula to insert the cannula in a patient and then inject the drug. The sequencing is again performed by a structure within the injector and which allows forward movement of the plunger only when the syringe has moved forwardly a sufficient extent.

Further examples of similar injectors including structures to ensure proper sequencing as aforesaid are to be found in U.S. Pat. No. 3,742,948, U.S. Pat. No. 2,752,918, U.S. Pat. No. 9,055,362, GB2388033, EP1364667-A2 and WO2005/002653-A1.

All of the prior art devices as described in the various specifications aforesaid use structures which are of varying degrees of complexity, to achieve a properly sequenced injection. The problem of this sequencing has been long understood—as evidenced by U.S. Pat. No. 2,752,918, filed on 1 May 1952.

Despite this, many injector designs ignore the problem of sequencing; WO03/013632 describes an medical injector having a mechanism which acts directly on a syringe plunger so as to drive the piston and thereby the entire syringe, needle and carrier forwards in relation to an external barrel by virtue of “the effective solidity of the dose” within a capsule. Thereafter, further pressing on the plunger continues to eject the dose, but as is well known and understood, medicament inevitably will leak through the needle during the first stage of operation when pressure is applied to the plunger to drive forwardly the entire syringe and carrier. Since liquid medicament is not truly solid, there will be unwanted loss of drug before the needle has entered the body.

As is clear from the wealth of prior art, there is a continuing need for a simple yet reliable and structurally compact medical injector that is able to achieve proper sequencing for an injection, of firstly inserting the needle fully and then dispensing the drug.

### BRIEF SUMMARY OF THE INVENTION

According to this invention there is provided a medical injector having a syringe body defining a bore, a needle fitted at the forward end of the syringe body, and a plunger projecting from the rearward end of the bore and a piston disposed in the bore so that medicament is expelled through the needle by depressing the end of the plunger remote from the piston, in which syringe the plunger has a protuberance disposed part way along the length thereof and in the region of the protuberance there is a slot through the plunger to allow the protuberance to move resiliently radially inwardly of the plunger, whereby the protuberance defines a stop position for the plunger on being moved into the bore by the application of axial pressure to the remote end of the plunger, so that when the plunger reaches the stop position an increased pressure is required to move the plunger deeper into the bore by moving the protuberance radially inwardly, thereby to expel medicament as aforesaid.

It will be appreciated that this invention allows the proper sequencing of an injection when the syringe is slidable relative to an external sleeve such that pressure applied to the rear end of a plunger will move the syringe and needle forwardly relative to the sleeve to allow the needle fully to penetrate a body whereafter increased force on the plunger will move the plunger past the stop position, the protuberance moving inwardly to permit this forward movement of the plunger, and dispense the medicament from the syringe body through the needle. Thus the resiliently deformable protuberance on the plunger enable the medical injector automatically to sequence a successful injection.

### BRIEF DESCRIPTION OF THE SEVERAL VIEWS OF THE DRAWINGS

The drawings show a specific embodiment of medical injector constructed and arranged in accordance with this invention, though solely by way of example. In the drawings, FIGS. 1A to 1J show a pre-filled syringe or a syringe to be filled from a vial of medicament, in varying stages of the use of the device. Specifically:

FIG. 1A is an isometric view of an axial section through the injector, in its initial condition;

FIG. 1B is an axial section of the injector of FIG. 1A, in the same condition as shown in FIG. 1A;

FIG. 1C shows the injector of FIGS. 1A and 1B in the course of filling from a vial, but not showing the vial;

FIG. 1D shows a first stage of the use of the injector with the needle protruding from the front thereof;

FIG. 1E shows the injector with the needle protruding fully but before the commencement of the dispensing medicament;

FIG. 1F shows the injector with the plunger fully forwardly;

FIG. 1G shows the injector after the completion of an injection and so with the sleeve blocked against rearward movement;

FIG. 1H is a partial axial section on an enlarged scale through the rear end of the syringe when in the condition shown in FIGS. 1B to 1E; and

FIG. 1J is a partial axial section on an enlarged scale through the forward part of the blocking member, when the syringe is in the condition shown in FIGS. 1B and 1C.

### DETAILED DESCRIPTION OF THE INVENTION

In the following description of this invention, the terms front, forward, and so on are used to refer to that end of the needle assembly whereat the sharp tip of the needle is located and also to the direction of insertion of the needle into a body. Conversely, the terms rear, rearwardly and so on are used to refer to the other end of the needle assembly, to which is connected other equipment such as a syringe or a blood collection system, and also to the direction of removal of a needle from a body.

The plunger of the preferred embodiment may have a plurality of protuberances having a slot associated therewith and extending through the plunger such that the protuberances may move resiliently inwardly.

The medical injector may be further provided with a safety arrangement which operates fully automatically and without the need for any operator intervention (i.e. passively) to furnish a sleeve over the exposed part of the used needle and block that sleeve in a fully protecting position, from which the sleeve cannot be withdrawn short of destroying the safety arrangement.

This embodiment of medical injector allows for use in much the same way as an unprotected hollow needle, except that injector provides protection to the needle tip before use, and wholly prevents access to the needle tip after use. In this way, protection may be afforded not just to the user, but also to others who could come into a risky situation with the used needle, such as waste disposal operators, cleaners, and so on.

The safety arrangement may include a blocking member at least a part of which projects forwardly from the syringe body, the blocking member being movable between a non-blocking position where the blocking member extends generally parallel to the needle axis and the sleeve may slide to its retracted position and a blocking position where the blocking member has moved from its non-blocking position so as to be disposed between the syringe body and a part of the sleeve, thereby blocking movement of the sleeve away from its protecting position. With such an arrangement, there is control means which releases the blocking member for movement from its non-blocking position to its blocking position on movement of the sleeve away from its initial position towards its retracted position, so that on subsequent movement of the sleeve to its protecting position the blocking member will thereafter block movement of the sleeve away from its protecting position.

On withdrawing the needle from a body, the safety arrangement operates fully automatically and without the need for any operator intervention (i.e. it operates passively), to furnish a sleeve over the exposed part of a needle and to block that sleeve in a fully protecting position, from which the sleeve cannot be withdrawn short of destroying the safety arrangement. Thus, an operator is automatically and effectively protected against needle-stick injuries, following the completion of a medical procedure using the needle, when equipped with the safety arrangement.

Preferably, the sleeve is translucent and a control member for the sleeve is of a high visibility material. In this way, the control member can also act as a visual indicator so that a user may readily see whether the assembly has been used and so should be discarded, because the control member will be visible at the forward end of the sleeve.

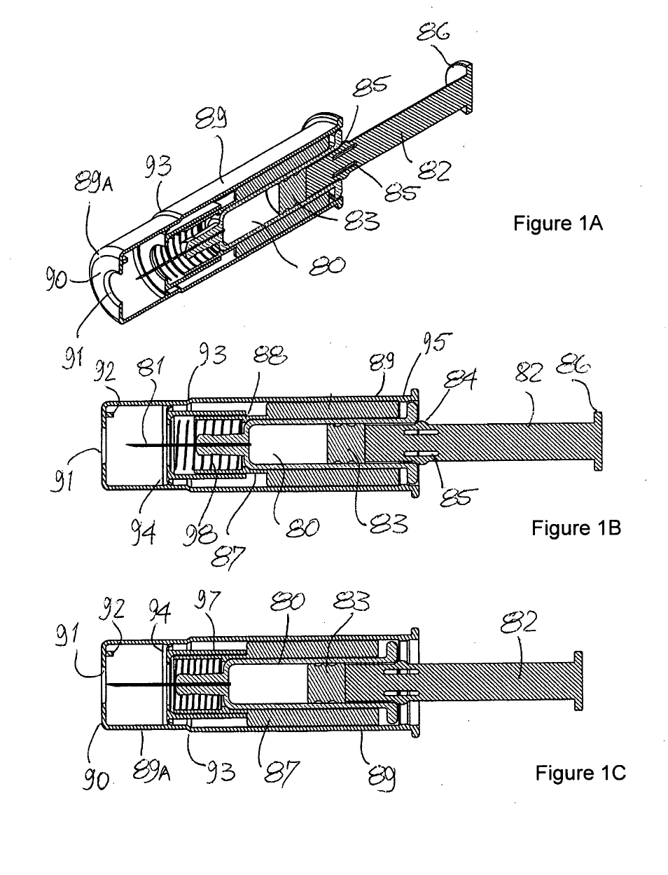

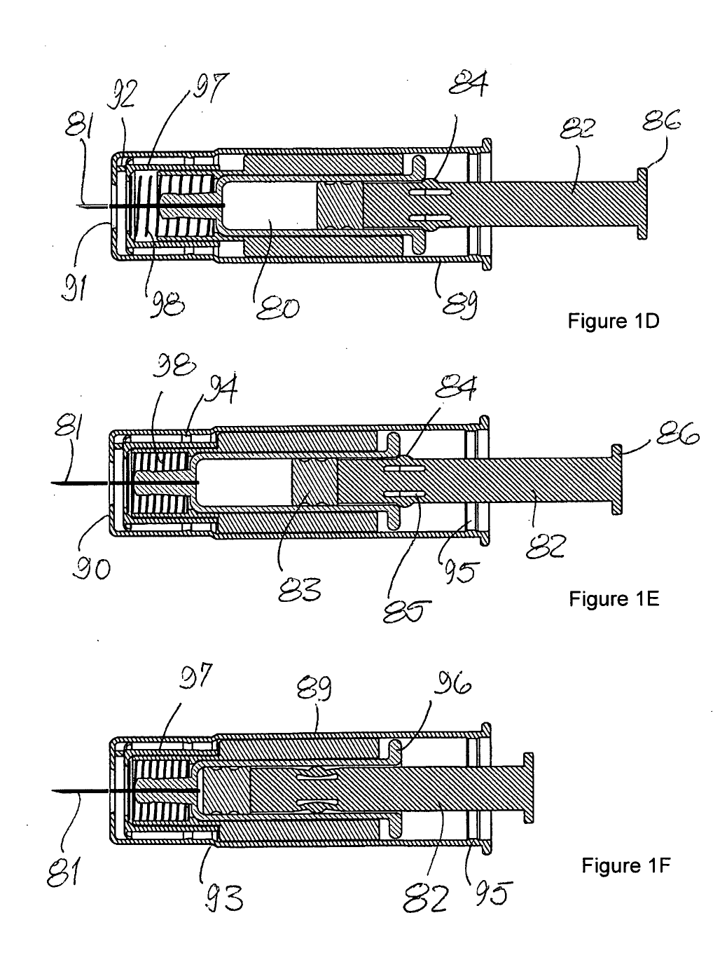

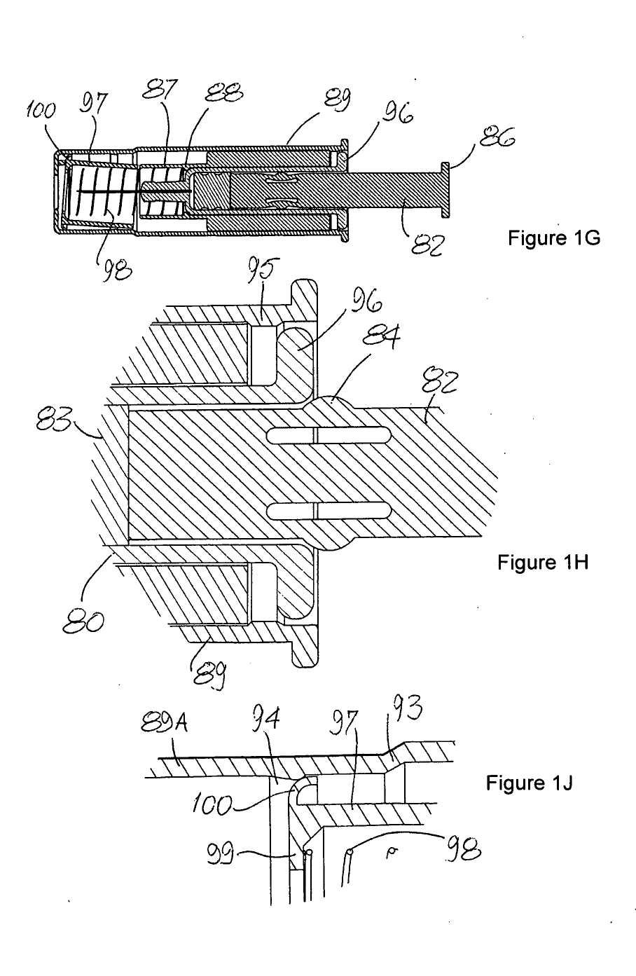

Referring now to FIGS. 1A to 1J of the drawings, these show a specific construction of a medical injector of this invention and using a syringe having a body 80 fitted with a needle 81 in the course of manufacture. The syringe has a plunger 82 with a piston 83 within the bore of the syringe, so that liquid may be drawn into the syringe through the needle 81 by withdrawing the plunger 82 from its fully inserted position, the medicament subsequently being expelled through the needle 81 by depressing that plunger.

The plunger 82 has an X-shaped cross-section and differs from the plunger of a conventional syringe in that the outer edge of each arm of the X-shaped cross-section is provided with a protuberance 84, disposed approximately one quarter of the way along the length of the plunger, from the piston end. In the region of each protuberance, the respective arm has a through-slot 85 to enable radially inward movement of the protuberances. The protuberances 84 define a stop position for the plunger on being moved into the bore by the application of axial pressure to the remote end 86 of the plunger. When the protuberances reach the rear end of the syringe body, an increased force is momentarily required to move the plunger deeper into the syringe body.

The syringe described above is provided with a safety arrangement which comprises a tubular support 87 having a bore in which the syringe body 80 is snugly received. The needle is thus indirectly carried by the support, through the syringe itself. Formed within that bore is an internal rib 88 which limits the movement of the syringe body into the bore. The part of the support 87 which overlies the syringe body has a greater wall thickness and slidably carries a sleeve 89. The forward end 89A of the sleeve has an internal radial flange 90 formed with a central hole 91 through which the needle 81 may project, the flange being provided with an upstand 92 which projects internally of the sleeve towards the syringe, the upstand having a relatively small arcuate extent, typically of only a few degrees. Partway along the length of the sleeve 89, an annular shoulder 93 is formed by a change in the internal and external diameters of the sleeve and between that shoulder 93 and the flange 90, there is formed an inwardly-projecting annular rib 94 (FIG. 1J). A further internal rib 95 (FIG. 1H) is formed at the rearward end of the sleeve, over which an out-turned flange 96 at the rear end of the syringe body must ride to permit the sleeve to slide rearwardly from that position shown in FIGS. 1A, 1B and 1H.

A tubular blocking member 97 is slidably carried on the forward end portion of the support 87 and is urged forwardly by a helical compression spring 98, acting between the internal rib 88 of the support and an internal flange 99 formed at the forward end of the blocking member 97. Externally, the blocking member 97 has at its forward end an outwardly-projecting flexible lip 100 slidable within the smaller diameter portion of the sleeve 89 but movable over the internal rib 94 of the sleeve only when an increased force is applied to the sleeve, relative to the blocking member. This is shown on an enlarged scale in FIG. 1J.

The starting position of the assembly is shown in FIGS. 1A, 1B, 1H and 1J in the case of a pre-filled syringe, with the safety arrangement set ready for use. Alternatively, the plunger will be fully forward to permit filling of the syringe in a case where an initial filling step is required.

If the syringe is to be filled, the nose part of a phial (not shown) of medicament is inserted into the hole 91 at the forward end of the sleeve 89 and is pushed gently on to the needle 81, moving the sleeve rearwardly with respect to the syringe by riding the further rib 95 of the sleeve over the out-turned flange 96 of the syringe body 80. During this, the blocking member 97 moves rearwardly, simultaneously with the sleeve, against the action of spring 98. Tile combined force of the spring 98 acting on the blocking member 97 and the force required to ride the further rib 95 over the out-turned flange 96 should be less than that required to move the lip 100 of the blocking member 97 over the rib 94 of the sleeve. As such, during the phial-filling operation, the blocking member 97 remains with its lip 100 rearward of rib 94 of the sleeve (FIG. 1C).

Following charging of the syringe and then the removal of the phial, the assembly is ready for performing an injection. The operator applies a gentle force on the remote end 86 of the plunger by applying a reaction to the sleeve 89 and this has the effect of moving the plunger forwardly until the protuberances 84 are about to enter the syringe body, and also of pulling the sleeve rearwardly, to cause the needle 81 to project from the forward end of the sleeve. However, this can be achieved only by having the lip 100 of the blocking member 97 ride over the rib 94 of the sleeve 89 and so moving forwardly towards the flange 90 of the sleeve, as shown in FIG. 1D. Rearward movement of the sleeve may continue until the shoulder 93 engages that part of the support having a thickened wall thickness as shown in FIG. 1E. The needle 81 is then projecting beyond the flange 90 to its greatest possible extent.

The assembly is used in this condition to perform an injection, firstly by pushing the needle 81 into a body at the injection site and then pushing the plunger fully forwardly, the protuberances 84 moving inwardly to permit this, as shown in FIG. 1F. The condition of FIG. 1E could instead be achieved by using the syringe with the connected assembly to perform a stabbing motion against a body, so that the engagement of the flange 90 at the forward end of the sleeve moves the sleeve rearwardly with respect to the syringe.

On removing the syringe assembly from a body, by pulling rearwardly on the plunger and releasing the sleeve, or by pulling on the sleeve and releasing the plunger, the spring 98 will cause relative separation of the forward end of the sleeve and the support 87, the spring acting on the flange 99 of the blocking member 97 to maintain contact between the forward end of the blocking member and upstand 92. Eventually, the separation will be so great that the blocking member comes free of the support 87 and the spring force acting on the blocking member will allow it to cant over so that its axis lies at an acute angle to the axis of the sleeve and support member—FIG. 1G. When in this position, the blocking member 97 lies between the flange 90 of the sleeve and the forward end of the support 87 and so physically blocks subsequent rearward movement of the sleeve 89, with respect to the support and syringe.

When in the setting of FIG. 1G, the needle is securely protected against exposure. Having regard to the tubular nature of the blocking member 97, a very high force must be applied to the sleeve 89 in order to expose the needle, in effect either destroying the sleeve or the blocking member.

The respective sleeves may be made transparent, translucent or provided with a transparent or translucent window. By manufacturing the blocking member from a strongly-coloured material, the position of the blocking member within the sleeve will readily be discernible. Then, when the safety device has been used and the blocking member is fully forward, this will immediately be apparent on looking at the assembly.

1. A medical injector having a syringe body defining a bore, a needle fitted at the forward end of the syringe body, and a plunger projecting from the rearward end of the bore and a piston disposed in the bore so that medicament is expelled through the needle by depressing the end of the plunger remote from the piston, in which syringe the plunger has a protuberance disposed part way along the length thereof and in the region of the protuberance there is a slot through the plunger to allow the protuberance to move resiliently radially inwardly of the plunger, whereby the protuberance defines a stop position for the plunger on being moved into the bore by the application of axial pressure to the remote end of the plunger, so that when the plunger reaches the stop position an increased pressure is required to move the plunger deeper into the bore by moving the protuberance radially inwardly, thereby to expel medicament as aforesaid.

2. A medical injector as claimed in claim 1, wherein there is a plurality of protuberances disposed in radial alignment with each other on the plunger.

3. A medical injector as claimed in claim 2, wherein each protuberance has a corresponding slot associated therewith and extending through the plunger.

4. A medical injector as claimed in claim 1 and including a safety device having a support defining a bore in which is received the syringe body and a sleeve mounted directly or indirectly on the support for sliding movement with respect thereto from an initial position where the sleeve covers at least the greater part of a carried needle to a retracted position where the tip of a carried needle and a part of the needle back from its tip is exposed, and then to a protecting position where the sleeve covers the needle tip and at least part of the needle back from its tip, and resilient means arranged to urge the sleeve towards its protecting position.

5. A medical injector as claimed in claim 4, wherein there is provided:
a blocking member at least a part of which projects forwardly from the syringe body, the blocking member being movable between a non-blocking position where the blocking member extends generally parallel to the needle axis and the sleeve may slide to its retracted position and a blocking position where the blocking member has moved from its non-blocking position so as to be disposed between the syringe body and a part of the sleeve, thereby blocking movement of the sleeve away from its protecting position; and
control means which releases the blocking member for movement from its non-blocking position to its blocking position on movement of the sleeve away from its initial position towards its retracted position, so that on subsequent movement of the sleeve to its protecting position the blocking member will thereafter block movement of the sleeve away from its protecting position.

6. A medical injector as claimed in claim 5, wherein the blocking member when in its blocking position extends at an acute angle to the needle axis.

7. A medical injector as claimed in claim 6, wherein the blocking member is tubular and when in its non-blocking position is generally co-axial with the sleeve and needle.

8. A medical injector as claimed in claim 4, wherein the sleeve is slidable on a support and the syringe body has an external surface which is received within the support.

9. A medical injector as claimed in claim 1, wherein the syringe body has the needle permanently secured to the forward end thereof.
